(19) United States
(12) Patent Application Publication
(10) Pub. No.: US 2024/0115629 A1
(43) Pub. Date: Apr. 11, 2024

(54) ORAL COMPOSITION

(71) Applicant: BLIS TECHNOLOGIES LIMITED, Dunedin (NZ)

(72) Inventors: John David Francis HALE, Dunedin (NZ); Rohit JAIN, Dunedin (NZ)

(21) Appl. No.: 18/546,044

(22) PCT Filed: Feb. 14, 2022
(86) PCT No.: PCT/IB2022/051269

§ 371 Date: Aug. 10, 2023

(30) Foreign Application Priority Data
Feb. 15, 2021 (AU) 2021900367

(51) Int. Cl. A61K 35/744; A61K 8/99; A61K 9/00; A61Q 11/00

(52) U.S. Cl. CPC A61K 35/744; A61K 8/99; A61K 9/0053; A61Q 11/00; A61K 2800/74; A61K 2800/92

(57) ABSTRACT

Described herein is an oral composition, such as a toothgel or a toothpaste, comprising Streptococcus salivarius, a viscosity modifier, a buffering agent, and a non-aqueous carrier. Advantageously, the oral composition may provide a desirable property, including a delayed release profile of the probiotic, improved colonisation in an oral cavity of a subject, improved stability and/or improved syneresis.

### FIELD OF THE INVENTION

The present invention generally relates to an oral composition comprising live probiotics. The invention also generally relates to methods of preparing oral compositions and methods of using the compositions to improve oral health.

### BACKGROUND TO THE INVENTION

Toothpastes and toothgels containing live probiotics are uncommon due to multiple technical challenges. These challenges include difficulties in keeping a probiotic alive in a toothpaste formulation, while providing for sustained release of the probiotic, and facilitating colonisation of the oral cavity of a subject. Moreover, each probiotic will respond differently within a composition. Traditional toothpaste and toothgel compositions typically include water, various gums and cellulose derivatives, sugars and sugar alcohols, and buffering agents which all serve as excellent growth media for probiotic bacteria. This excess growth results in overuse of nutrients subsequently leading to a death phase, which in turn results in a reduction in cell viability and therefore an ineffective product. Additionally, due to their aqueous nature and ingredients, aqueous toothpastes and gels require the addition of preservatives to prevent growth of pathogens. These preservatives are typically non-selective and lead to probiotic cell death, and therefore loss in efficacy within a short time. The presence of strong surfactants in typical foaming toothpastes can also affect the viability of the probiotic cells in situ in the oral cavity making the probiotics ineffective. A composition containing an aqueous vehicle is therefore neither clinically efficacious nor commercially viable as an oral hygiene formulation for probiotics.

Non-aqueous oil or lipid based probiotic formulations provide better shelf-life stability due to lack of moisture but pose other challenges including probiotic release rate issues. A probiotic dispersed in a viscous matrix of oil and viscosity modifiers may become trapped within the matrix leading to insignificant or insufficient release of the probiotic from the matrix. This in turn contributes to no, or low availability of probiotic and low colonisation efficacy in vivo. Conversely, without an oil or aqueous and oil formulation a probiotic may be released instantly from the release medium. This will not allow enough time for the probiotic to be retained in the oral cavity to achieve colonisation.

Another challenge that may be encountered when preparing a non-aqueous oil or lipid formulation is syneresis. Syneresis is the leakage of oil from the matrix due to contraction of the gel upon storage. To overcome syneresis, it is often necessary to include a high proportion of gelling agents (e.g. hydrogenated lipids, waxes) to prevent syneresis. However, the addition of a high proportion of gelling agents may result in highly viscous systems that are difficult to dispense as a toothpaste, or do not allow for release of probiotic.

As will be appreciated, a composition in a liquid suspension is not suitable for toothpaste application. However, a highly viscous composition is also not suitable.

Some formulations require heating steps to allow the mixing of the probiotic as the formulations solidify at room temperature. Solidification issues are often addressed using long duration high shear homogenisation to obtain uniform mixing. These conditions pose a risk of killing the probiotic or generating weaker probiotic cells which may affect long term stability and efficacy of the formulation.

WO2017/195074 A1 (OraHealth Corp.) describes a lipid-based mixture for mouth coating that may contain probiotics. The composition may be a gel but WO2017/195074 A1 does not describe a paste. WO2012/097429 A1 (Viva Pharmaceutical Inc) describes a soft gel capsule containing probiotic bacteria. WO2010/054439 A1 (Unistraw Patent Holdings Limited) describes probiotic bacteria embedded within a matrix that may be a non-aqueous, oil-based matrix.

Seok et al. “Formulating a probiotic toothpaste for vitamin B6 delivery system”, Journal of International Research in Medical and Pharmaceutical Sciences, 2018 (13) describes a probiotic toothpaste formulation to deliver vitamin B6 to the oral cavity. The probiotic element is TheraBreath Oral Care Probiotics Citrus, which contains Streptococcus salivarius strains K12 and M18 (BLIS K12 and M18) cells. Seok et al. focuses on transmucosal delivery of vitamin B6 in the oral cavity. The compositions comprise various ingredients incompatible with probiotics including glycerine, hydrogen peroxide and water.

CN111558033 describes oral cleaning compositions comprising at least a proteolytic enzyme, a non-proteolytic enzyme, BLIS K12 and M18, and at least one Lactobacillus spp. These compositions also comprise glycerol and water which are incompatible with probiotics.

While these documents contemplate oral gels containing probiotics, the inventors are not aware of any commercial oral compositions being developed, based on these patent applications, that exhibit an adequate release profile balanced with probiotic viability, or that contain Streptococcus salivarius.

WO2001027143 (BLIS Technologies Limited) describes Streptococcus salivarius strains K12 and K30 on deposit at Deutsche Sammlung von Mikroorganismen und Zellkulturen GmbH, Braunschweig, Germany, under accession numbers DSM 13084 and DSM 13085 respectively. Also described is the use of these probiotic strains to prevent or treat infections of the upper respiratory tract including the mouth. Conditions to be treated include streptococcal sore throats and dental caries. WO2005007178 (BLIS Technologies Limited) further describes use of Streptococcus salivarius K12 and K30 for the treatment of halitosis.

WO2003070919 (BLIS Technologies Limited) describes Streptococcus salivarius strain Mia (also known as M18) on deposit at Deutsche Sammlung von Mikroorganismen und Zellkulturen GmbH, Braunschweig, Germany, under accession No. DSM 14685. Also described is the use of this probiotic strain for its anti-mutans streptococci (MS) activity, in particular its activity against S. mutans and S. sobrinus, and the treatment of dental caries. While oral formulations such as food, drink, syrup, gargle, toothpaste, lozenges, and mouthsprays are mentioned in WO2001027143, WO2003070919 and WO2005007178, to date, no commercial toothpaste or gel formulation has been developed containing these Streptococcus salivarius. The lack of a commercial product reflects the difficulties in producing a probiotic toothpaste or gel composition that exhibits desirable release properties for the probiotic, while maintaining probiotic viability.

There is an ongoing need for a probiotic oral composition that is stable and provides desired release and colonisation characteristics. It is an object of the present invention to go some way to meeting this need; and/or to at least provide the public with a useful choice.

Other objects of the invention may become apparent from the following description which is given by way of example only.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention provides an oral composition comprising a Streptococcus salivarius, a viscosity modifier, a buffering agent, and a non-aqueous carrier, wherein release of the Streptococcus salivarius from the composition at 15 minutes after being administered to an oral cavity is in the range of from about 35% to about 95%.

In a second aspect, the present invention provides a toothpaste composition comprising a Streptococcus salivarius, a buffering agent, a viscosity modifier, an emulsifier, and a non-aqueous carrier, wherein the buffering agent is calcium carbonate, and the viscosity modifier is hydrophobic silica.

In a third aspect, the present invention provides a toothpaste composition comprising a Streptococcus salivarius, calcium carbonate, hydrophobic silica, an emulsifier, and a non-aqueous carrier, wherein release of the Streptococcus salivarius from the composition at 15 minutes after being administered to an oral cavity is in the range of from about 35% to about 95%.

In a fourth aspect, the present invention provides a method of manufacturing a composition according to the first, second, or third aspect, comprising the steps of:
a) mixing a non-aqueous carrier and an emulsifier,
b) dispersing Streptococcus salivarius and a buffering agent in the mixture from step a),
c) adding a viscosity modifier to the mixture from step b), and
d) homogenising the mixture from step c) to provide the composition.

In a fifth aspect, the present invention provides a method of manufacturing a composition according to the first, second, or third aspect, comprising the steps of:
a) heating a non-aqueous carrier,
b) adding a viscosity modifier to the heated non-aqueous carrier,
c) allowing the mixture from step b) to partially cool and adding an emulsifier to the mixture,
d) allowing the mixture from step c) to cool further and adding a Streptococcus salivarius to the mixture, and
e) homogenising the mixture from step d) to provide the composition.

In a sixth aspect, the present invention provides a method of improving oral health in a subject, comprising applying a composition according to the first, second, or third aspect to the oral cavity of the subject.

In a seventh aspect, the present invention provides a method of delivering a probiotic to the oral cavity of a subject, comprising administering an oral composition according to the first, second, or third aspect to the oral cavity of the subject.

The following embodiments and preferences may relate alone or in any combination of any two or more to any of the above aspects.

In some embodiments, release of the Streptococcus salivarius from the composition at 30 minutes after being administered to an oral cavity is in the range of from about 50% to about 98%.

Preferably, the Streptococcus salivarius is Streptococcus salivarius M18, Streptococcus salivarius K12, or a combination thereof.

Preferably, the composition comprises the Streptococcus salivarius in an amount of about 1×10⁵ to about 1×10¹² cfu/g. More preferably, the composition comprises the Streptococcus salivarius in an amount of about 1×10⁷ to about 1×10¹⁰ cfu/g.

In some embodiments, the composition comprises the viscosity modifier in an amount of about 1 to about 15% w/w.

In some embodiments, the viscosity modifier is selected from the group consisting of hydrophobic silica, wax, ethyl cellulose, stearic acid, tapioca starch, xanthan gum, Carbopol 974p (carbomer), and a combination of any two or more thereof. Preferably, the viscosity modifier is hydrophobic silica, ethyl cellulose, wax, or a combination of two or more thereof.

Preferably, the wax is selected from group consisting of white beeswax, yellow beeswax, paraffin wax, jojoba wax, microcrystalline wax, shea butter, cocoa butter, and a combination of any two or more thereof.

In some embodiments, the composition comprises the buffering agent in an amount of about 1 to about 30% w/w. In some embodiments, the composition comprises the buffering agent in an amount of about 10 to about 20% w/w. In some embodiments, the composition comprises the buffering agent in an amount of about 15% w/w.

In some embodiments, the buffering agent is selected from the group consisting of calcium carbonate, sodium bicarbonate, sodium chloride, sodium or potassium phosphate salts, magnesium carbonate, hydrated aluminium oxides, bentonite clay, kaolin clay, and a combination of any two or more thereof. In some embodiments, the sodium or potassium phosphate salt is selected from sodium hydrogen phosphate, sodium dihydrogen phosphate, dipotassium hydrogen phosphate, potassium dihydrogen phosphate, and a combination of any two or more thereof.

In some embodiments, the non-aqueous carrier is selected from the group consisting of a medium chain triglyceride, triacetin, ethyl oleate, glycerol, propylene glycol, vegetable oil, and a combination of any two or more thereof. In some embodiments, the non-aqueous carrier is selected from the group consisting of a medium chain triglyceride, triacetin, ethyl oleate, propylene glycol, vegetable oil, and a combination of any two or more thereof. Preferably, the medium chain triglyceride is a caprylic/capric triglyceride. Preferably, the vegetable oil is selected from the group consisting of sunflower oil, canola oil, soybean oil, olive oil, and a combination of any two or more thereof.

In some embodiments, the composition further comprises an emulsifier, an antibacterial agent, a sweetener, a flavouring agent, a fluoride source, an additional probiotic, a foaming agent, a colourant, an abrasive, a whitening agent, a tooth sensitivity agent, an antioxidant, a remineralisation agent, or a combination of any two or more thereof.

In some embodiments, the emulsifier is selected from the group consisting of polysorbate 80, sorbitan oleate, egg lecithin, soybean lecithin, polyoxyl 35 castor oil, and a combination of any two or more thereof.

In some embodiments, the antibacterial agent is selected from the group consisting of xylitol, erythritol, Manuka honey, Kamahi honey, propolis, tea tree oil, and a combination of any two or more thereof.

In some embodiments, the sweetener is selected from the group consisting of mogroside sweetener, sucralose, stevia, aspartame, saccharin, thaumatin, sorbitol, maltodextrin, isomalt, sucrose, honey, and a combination of any two or more thereof.

In some embodiments, the fluoride source is selected from the group consisting of sodium monofluorophosphate, sodium fluoride, stannous fluoride, and a combination of any two or more thereof.

In some embodiments, the additional probiotic is selected from the group consisting of a Lactobacillus spp., a Bifidobacterium spp., a Streptococcus spp., a Saccharomyces spp., and a combination of any two or more thereof. In some embodiments, the additional probiotic is selected from the group consisting of a Lactobacillus spp., a Bifidobacterium spp., a Streptococcus spp., a Saccharomyces spp., a Limosilactobacillus spp., a Lacticaseibacillus spp., a Ligilactobacillus spp., and a combination of any two or more thereof.

In some embodiments, the abrasive is selected from the group consisting of silica, calcium carbonate, sodium bicarbonate, fluoride, sodium chloride, phosphate salts, magnesium carbonate, hydrated aluminum oxides, clay, activated charcoal, tetrasodium pyrophosphate, disodium pyrophosphate, and a combination of any two or more thereof. In some embodiments, the abrasive is selected from the group consisting of silica, calcium carbonate, sodium bicarbonate, sodium chloride, phosphate salts, magnesium carbonate, hydrated aluminum oxides, clay, activated charcoal, tetrasodium pyrophosphate, disodium pyrophosphate, and a combination of any two or more thereof.

In some embodiments, the composition has a shelf-life of at least 2 months at 25° C./60% RH. In some embodiments, the composition has a shelf-life of at least 6 months at 25° C./60% RH. In some embodiments, the composition has a shelf-life of at least 30 months at 25° C./60% RH.

In some embodiments, the composition has a viscosity of greater than 1,900,000 cp at 25° C. In some embodiments, the composition has a viscosity of from about 20,000 to about 2,000,000 cp at 25° C. In some embodiments, the composition has a viscosity of from about 20,000 to about 500,000 cp at 25° C.

In some embodiments, the composition is a paste or a gel. In some embodiments, the composition is a gel and has a viscosity of about 35,000 to about 65,000 cp at 25° C. In some embodiments, the composition is a toothpaste. Preferably, the paste has a viscosity of about 70,000 to about 100,000 cp at 25° C.

In some embodiments, the composition has a syneresis ratio of about 10% to about 40% after being centrifuged at 13,000 rpm for 5 minutes at 25° C. In some embodiments, the composition has a syneresis ratio of about 20% to about 40% after being centrifuged at 13,000 rpm for 5 minutes at 25° C. In some embodiments, the composition has a syneresis ratio of about 10% to about 20%, or about 13% to about 18% after being centrifuged at 13,000 rpm for 5 minutes at 25° C.

In some embodiments, the composition comprises: about 1×10⁵ to about 1×10¹⁰ cfu/g of Streptococcus salivarius M18, about 1 to about 30% w/w of calcium carbonate, about 1 to about 15% w/w of hydrophobic silica, and about 0.1 to about 5% w/w of the emulsifier.

In some embodiments, the composition comprises: about 1×10⁶ to about 1×10¹⁰ cfu/g of Streptococcus salivarius M18, about 10 to about 20% w/w of calcium carbonate, about 3 to about 8% w/w of hydrophobic silica, and about 1 to about 2% w/w of the emulsifier.

In some embodiments, the composition comprises: about 1×10⁸ to about 1×10⁹ cfu/g of Streptococcus salivarius M18, about 15% w/w of calcium carbonate, about 4 to about 6% w/w of hydrophobic silica, and about 1% w/w of the emulsifier.

In some embodiments the composition comprises: about 1×10⁸ to about 1×10⁹ cfu/g of Streptococcus salivarius M18, about 20% w/w of calcium carbonate, about 6 to about 10% w/w of hydrophobic silica, and about 2.5% w/w of the emulsifier.

In some embodiments the composition comprises: about 1×10⁸ to about 1×10⁹ cfu/g of Streptococcus salivarius M18, about 18 to about 22% w/w of calcium carbonate, about 6 to about 10% w/w of hydrophobic silica, and about 2.5% w/w of the emulsifier.

In some embodiments, the composition comprises:
about 1×10⁸ to about 1×10⁹ cfu/g of Streptococcus salivarius M18,
about 20% w/w of buffering agent,
about 1 to about 10% w/w of each viscosity modifier,
about 2.5% w/w of the emulsifier,
about 0.76% w/w of the fluoride source,
about 2 to about 8% w/w of the antibacterial agent,
about 0.01 to about 5% of each flavouring agent, and
about 3% w/w sweetener
based on the total weight of the composition.

In some embodiments, the composition comprises:
about 1×10⁸ to about 1×10⁹ cfu/g of Streptococcus salivarius M18,
about 20% w/w of calcium carbonate,
about 4 to about 8% w/w of hydrophobic silica,
about 2 to about 8% of xanthan gum,
about 2.5% w/w of polysorbate 80
about 0.76% w/w of the fluoride source,
about 2 to about 8% w/w of xylitol,
about 2 to about 3% w/w of spearmint flavouring oil,
about 2 to about 3% w/w of peppermint flavouring oil,
about 0.3% w/w of Smoothenol Flavour 30712 (comprising maltodextrin, gum arabic and triacetin), and
about 3% w/w of Synergy Powder Sweetener PF 7513 (comprising maltodextrin and natural flavouring)
based on the total weight of the composition.

In some embodiments, release of the Streptococcus salivarius from the composition at 15 minutes after being administered to an oral cavity is in the range of from about 35% to about 95%.

In some embodiments, the method is for reducing dental caries in a subject, removing and/or preventing stains and/or plaque from the teeth of the subject, strengthening the enamel on the teeth of the subject, treating and/or preventing gingivitis, assisting gum healing, and/or preventing halitosis.

In some embodiments, the probiotic at least partially colonises the oral cavity. Preferably, the probiotic is a Streptococcus salivarius, such as M18, K12, or a combination thereof.

The invention may also be said broadly to consist in the parts, elements and features referred to or indicated in the specification of the application, individually or collectively, in any or all combinations of two or more of said parts, elements or features, and where specific integers are mentioned herein which have known equivalents in the art to which the invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Although the present invention is broadly as defined above, those persons skilled in the art will appreciate that the invention is not limited thereto, and that the invention also includes embodiments of which the following description gives examples.

### BRIEF DESCRIPTION OF THE FIGURES

The present invention will be described with reference to the accompanying figures, in which:

FIG. 1 shows samples of comparative formulations (from left to right) C-1, C-2 and C-3.

FIG. 2 shows a sample of formulation C-1 after being kept in cold storage.

FIG. 3 is a graph showing accelerated syneresis of formulations A-4, C-1 and C-2.

FIG. 3B is a graph showing accelerated syneresis of formulations A-4, C-1, C-2, B-6, and B-7.

FIG. 4 is a graph showing the results of a release study using formulation A-1.

FIG. 5 is a graph showing the results of a release study using formulation A-2.

FIG. 6 is a graph showing the results of a release study using formulation A-3.

FIG. 7 is a graph showing the results of a release study using a BLIS M18 lozenge, and formulations A-1 to A-4, A-7 and C-1 to C-3.

FIG. 7B is a graph showing the results of a release study using a BLIS M18 lozenge, and formulations A-1 to A-4, A-7, C-1 to C-3, the formulation of CN111558033, and formulations B-6, and B-7.

FIG. 8 is a bar graph showing colonisation in subjects with M18 following administration of a lozenge containing M18 and K12.

FIG. 9 is a bar graph showing colonisation in subjects with M18 following brushing with toothpaste A-4.

FIG. 10 is a graph showing the results of a stability study with the formulation of Seok et al.

FIG. 11 is a graph showing the results of a stability study with the formulation of CN111558033.

FIG. 12 is graph showing stability data of formulations A-4, A-6, B-8, B-9, B-10, B-11, B-3, B-6, and C-2.

FIG. 13 is a graph showing inhibitory activity of BLIS M18 in a raw ingredient compared to in a toothpaste formulation.

### DETAILED DESCRIPTION OF THE INVENTION

Described herein is an oral composition, such as a toothgel or a toothpaste, comprising Streptococcus salivarius, a viscosity modifier, a buffering agent, and a non-aqueous carrier. Advantageously, the oral composition is carefully formulated to provide controlled release properties resulting in a delayed release profile of the probiotic, which still allows for colonisation in an oral cavity of a subject. The composition also exhibits long-term stability and/or limited syneresis. Long-term stability is the ability of the composition to ensure an efficacious viable number of probiotics under standard storage and humidity conditions. Syneresis is the contraction of a gel accompanied by the separating out of liquid.

### Definitions

The term “comprising” as used in this specification and claims means “consisting at least in part of”. When interpreting each statement in this specification and claims that includes the term “comprising”, features other than that or those prefaced by the term may also be present. Related terms such as “comprise”, “comprised” and “comprises” are to be interpreted in the same manner.

As used herein the term “and/or” means “and” or “or”, or both.

As used herein “(s)” following a noun means the plural and/or singular forms of the noun. The general chemical and biological terms used, for example, in the formulae herein have their usual meanings.

As used herein, the term “gel” means a solid or semisolid system of at least two constituents, consisting of a condensed mass enclosing and interpenetrated by a liquid (e.g. a non-aqueous carrier).

As used herein, the term “paste” means a semisolid dosage form, containing a large proportion of solids (e.g. about 20-50% by weight) finely dispersed into a suitable carrier.

The term “subject” as used herein refers to a mammal, including humans, dogs, cats, horses, sheep, cows and other domestic and farm animals.

The unit “cfu/g” means colony-forming units per gram.

### Probiotic

Streptococcus salivarius are Gram-positive bacteria that predominantly colonise the human oral cavity, specifically the tongue, and are the dominant commensal species. They are highly investigated for use as probiotic bacteria. A number of Streptococcus salivarius species have been commercialised by BLIS Technologies with trade names BLIS M18 and BLIS K12 for oral and dental health.

A range of Streptococcus salivarius strains suitable for use in the invention are known in the art. Streptococcus salivarius K12 was deposited with Deutsche Sammlung von Mikroorganismen und Zellkulturen GmbH, Mascheroder Weg 1 b, D-38124, Braunschweig, Germany on 8 Oct. 1999, and assigned Accession No. DSM 13084. Streptococcus salivarius K12 is described in WO2001027143 supra, incorporated herein by reference. Streptococcus salivarius M18 was deposited at Deutsche Sammlung von Mikroorganismen und Zellkulturen GmbH, Mascheroder Weg 1 b, D-38124, Braunschweig, Germany on Dec. 12, 2001, and assigned Accession No. DSM 14685. Streptococcus salivarius M18 is described in WO2003070919 supra, incorporated herein by reference.

In some embodiments, the Streptococcus salivarius is a live probiotic. In some embodiments, the Streptococcus salivarius strain is M18, K12, or a combination thereof.

In some embodiments, the composition comprises about 1×10⁵ to about 1×10¹² cfu/g of the Streptococcus salivarius. In some embodiments, the composition comprises about 1×10⁶ to about 1×10¹⁰ cfu/g, about 1×10⁷ to about 1×10⁹ cfu/g, about 1×10⁷ to about 1×10¹⁰ cfu/g, about 1×10⁸ to about 1×10¹⁰ cfu/g, or about 1×10⁸ to about 1×10⁹ cfu/g of the Streptococcus salivarius. In some embodiments, the composition comprises about 1×10⁹ cfu/g of the Streptococcus salivarius. In some embodiments, the composition comprises about 2×10⁹ cfu/g of the Streptococcus salivarius.

In some embodiments, where multiple strains of Streptococcus salivarius are present, the composition comprises about 1×10⁵ to about 1×10¹² cfu/g of each strain of Streptococcus salivarius. In some embodiments, the composition comprises about 1×10⁶ to about 1×10¹⁰ cfu/g, about 1×10⁷ to about 1×10⁹ cfu/g, about 1×10⁷ to about 1×10¹⁰ cfu/g, about 1×10⁸ to about 1×10¹⁰ cfu/g, or about 1×10⁸ to about 1×10⁹ cfu/g of each strain of the Streptococcus salivarius. In some embodiments, the composition comprises about 1×10⁹ cfu/g of each strain of the Streptococcus salivarius. In some embodiments, the composition comprises about 2×10⁹ cfu/g of each strain of the Streptococcus salivarius.

In some embodiments, the composition comprises about 1×10⁵ to about 1×10¹² cfu/g of the Streptococcus salivarius M18. In some embodiments, the composition comprises about 1×10⁶ to about 1×10¹⁰ cfu/g, about 1×10⁷ to about 1×10⁹ cfu/g, about 1×10⁷ to about 1×10¹⁰ cfu/g, about 1×10⁸ to about 1×10¹⁰ cfu/g, or about 1×10⁸ to about 1×10⁹ cfu/g of the Streptococcus salivarius M18. In some embodiments, the composition comprises about 1×10⁹ cfu/g of the Streptococcus salivarius M18. In some embodiments, the composition comprises about 2×10⁹ cfu/g of the Streptococcus salivarius M18.

In some embodiments, the composition comprises about 1×10⁵ to about 1×10¹² cfu/g of the Streptococcus salivarius K12. In some embodiments, the composition comprises about 1×10⁶ to about 1×10¹⁰ cfu/g, about 1×10⁷ to about 1×10⁹ cfu/g, about 1×10⁷ to about 1×10¹⁰ cfu/g, about 1×10⁸ to about 1×10¹⁰ cfu/g, or about 1×10⁸ to about 1×10⁹ cfu/g of the Streptococcus salivarius K12. In some embodiments, the composition comprises about 1×10⁹ cfu/g of the Streptococcus salivarius K12. In some embodiments, the composition comprises about 2×10⁹ cfu/g of the Streptococcus salivarius K12.

In some embodiments, the composition comprises about 1×10⁵ to about 1×10¹² cfu/g of the Streptococcus salivarius M18 and about 1×10⁵ to about 1×10¹² cfu/g of the Streptococcus salivarius K12. In some embodiments, the composition comprises about 1×10⁶ to about 1×10¹⁰ cfu/g, about 1×10⁷ to about 1×10⁹ cfu/g, about 1×10⁷ to about 1×10¹⁰ cfu/g, about 1×10⁸ to about 1×10¹⁰ cfu/g, or about 1×10⁸ to about 1×10⁹ cfu/g of the Streptococcus salivarius M18 and about 1×10⁶ to about 1×10¹⁰ cfu/g, about 1×10⁷ to about 1×10⁹ cfu/g, about 1×10⁷ to about 1×10¹⁰ cfu/g, about 1×10⁸ to about 1×10¹⁰ cfu/g, or about 1×10⁸ to about 1×10⁹ cfu/g of the Streptococcus salivarius K12.

In some embodiments, the composition further comprises an additional probiotic. Suitable probiotics include, but are not limited to, Lactobacillus spp. (e.g. L. acidophilus, L. reuteri, L. rhamnosus, or L. salivarius), Bifidobacterium spp. (e.g. B. bifidum, B. longum, or B. lactis BB12), Streptococcus spp. (e.g. S. oralis, S. uberis, or Streptococcus salivarius K12), and Saccharomyces spp. (e.g. S. boulardii or S. cerevisiae). Note Lactobacillus reuteri is now known as Limosilactobacillus reuteri, Lactobacillus rhamnosus is now known as Lacticaseibacillus rhamnosus, and Lactobacillus salivarius is now known as Ligilactobacillus salivarius. Accordingly, suitable probiotics include, but are not limited to, Lactobacillus spp. (e.g. L. acidophilus), Bifidobacterium spp. (e.g. B. bifidum, B. longum, or B. lactis BB12), Streptococcus spp. (e.g. S. oralis, S. uberis, or Streptococcus salivarius K12), Saccharomyces spp. (e.g. S. boulardii or S. cerevisiae), Limosilactobacillus spp. (e.g. L. reuteri), Lacticaseibacillus spp. (e.g. L. rhamnosus), or Ligilactobacillus spp. (e.g. L. salivarius).

### Viscosity Modifier

The oral composition comprises a viscosity modifier. Advantageously, the viscosity modifier may be used to control the stability of the composition and/or to modulate the release profile of the probiotic, e.g. when administered to an oral environment.

Suitable viscosity modifiers include, but are not limited to, hydrophobic silica, waxes (such as white beeswax, yellow beeswax, paraffin wax, jojoba wax, microcrystalline wax), shea butter, cocoa butter, ethyl cellulose, stearic acid, tapioca starch, xanthan gum, Carbopol 974p (carbomer), peanut butter, or a combination of any two or more thereof. In some embodiments, the viscosity modifier is ethyl cellulose. In some embodiments, the viscosity modifier is a combination of ethyl cellulose and a wax, such as beeswax. Preferably, the viscosity modifier is hydrophobic silica (e.g. Aerosil R972®).

In some embodiments, the oral composition comprises the viscosity modifier in an amount of about 1 to about 15% w/w based on the total weight of the composition. For example, the composition may comprise the viscosity modifier in an amount of about 4 to about 15%, or about 4 to about 10%, or about 3 to about 8%, or about 4 to about 6%, or about 4 to about 8% w/w based on the total weight of the composition. In various embodiments, the composition may comprise the viscosity modifier in an amount of about 6 to about 10%, or about 7 to about 9% w/w based on the total weight of the composition. For example, the composition may comprise the viscosity modifier in an amount of about 1%, about 2%, about 3%, about 4%, about 5%, about 6%, about 7%, about 8%, about 9%, about 10%, about 11%, about 12%, about 13%, about 14%, or about 15% w/w based on the total weight of the composition. In some embodiments, the composition comprises the viscosity modifier (e.g. hydrophobic silica) in an amount of about 6% w/w based on the total weight of the composition. In some embodiments, the composition comprises the viscosity modifier (e.g. hydrophobic silica) in an amount of about 7% w/w based on the total weight of the composition. In some embodiments, the composition comprises the viscosity modifier (e.g. hydrophobic silica) in an amount of about 8% w/w based on the total weight of the composition. In some embodiments, the composition comprises the viscosity modifier (e.g. xanthan gum) in an amount of about 5% w/w based on the total weight of the composition. In some embodiments, the composition comprises the viscosity modifier (e.g. xanthan gum) in an amount of about 2% w/w based on the total weight of the composition.

In some embodiments, where multiple viscosity modifiers are present, the oral composition comprises each viscosity modifier in an amount of about 1 to about 15% w/w based on the total weight of the composition. For example, the composition may comprise each viscosity modifier in an amount of about 1 to about 10%, about 1 to about 15%, about 1 to about 8%, about 2 to about 15%, about 2 to about 10%, about 2 to about 15%, about 2 to about 8%, about 4 to about 15%, or about 4 to about 10%, or about 3 to about 8%, or about 4 to about 6%, or about 4 to about 8% w/w based on the total weight of the composition. In various embodiments, the composition may comprise each viscosity modifier in an amount of about 6 to about 10%, or about 7 to about 9% w/w based on the total weight of the composition. For example, the composition may comprise each viscosity modifier in an amount of about 1%, about 2%, about 3%, about 4%, about 5%, about 6%, about 7%, about 8%, about 9%, about 10%, about 11%, about 12%, about 13%, about 14%, or about 15% w/w based on the total weight of the composition. In some embodiments, the viscosity modifiers are hydrophobic silica and xanthan gum.

### Buffering Agent

The oral composition may comprise a buffering agent. Advantageously, the buffering agent may promote growth of the probiotic in the oral composition, modulate syneresis of the composition and/or act as an abrasive.

Suitable buffering agents include, but are not limited to, calcium carbonate, sodium bicarbonate, sodium chloride, sodium or potassium phosphate salts (such as sodium hydrogen phosphate, sodium dihydrogen phosphate, dipotassium hydrogen phosphate, and potassium dihydrogen phosphate), magnesium carbonate, hydrated aluminum oxides, bentonite clays, kaolin clay, urea, or a combination of any two or more thereof. Preferably, the buffering agent is calcium carbonate. For long term stability, the applicants have surprisingly found that insoluble (non-precipitated) calcium carbonate demonstrates good microbial stability for at least 6 months. Without wishing to be bound by theory, the inventors believe precipitated or slightly water soluble calcium carbonate may be more hygroscopic, thus affecting stability. In various embodiments, the calcium carbonate is insoluble calcium carbonate. Insoluble calcium carbonate may be sourced from Pure Nature or Sigma.

In some embodiments, the oral composition comprises the buffering agent in an amount of about 1 to about 30% w/w based on the total weight of the composition. For example, the composition may comprise the buffering agent in an amount of about 5 to about 25%, or about 10 to about 20% w/w based on the total weight of the composition. In various embodiments, the composition may comprise the buffering agent in an amount of about 15 to about 25%, or about 18 to about 22% w/w based on the total weight of the composition. In various embodiments, the composition comprises the buffering agent in an amount of about 12 to about 18% w/w based on the total weight of the composition. In some embodiments, the composition comprises the buffering agent in an amount of about 5%, about 10%, about 15%, about 20%, about 25%, or about 30% w/w based on the total weight of the composition. In some embodiments, the buffering agent (e.g. calcium carbonate) is about 15% w/w based on the total weight of the composition. In some embodiments, the composition comprises buffering agent (e.g. calcium carbonate) in an amount of about 20% w/w based on the total weight of the composition.

### Non-Aqueous Carrier

The oral composition comprises a non-aqueous carrier. Suitable non-aqueous carriers include, but are not limited to, medium chain triglycerides, triacetin, ethyl oleate, glycerol, propylene glycol, vegetable oil, polyethylene glycol, or a combination of any two or more thereof. In various embodiments, the non-aqueous carrier is selected from medium chain triglycerides, triacetin, ethyl oleate, propylene glycol, vegetable oil, polyethylene glycol, or a combination of any two or more thereof. Preferably, the medium chain triglyceride is a caprylic/capric triglyceride, such as Miglyol 812N (triglyceride ester of saturated coconut/palm-kernel oil derived caprylic and capric fatty acids and plant derived glycerol). In some embodiments, the vegetable oil is selected from the group consisting of sunflower oil, canola oil, soybean oil, olive oil, and a combination of any two or more thereof.

In some embodiments, the oral composition comprises the non-aqueous carrier in a quantity sufficient (q.s.) amount, i.e. an amount to bring the total w/w % of the composition to 100%. In some embodiments, the oral composition comprises the non-aqueous carrier in an amount of about 30 to about 95% w/w based on the total weight of the composition. In some embodiments, the oral composition comprises the non-aqueous carrier in an amount of about 55 to about 95% w/w based on the total weight of the composition. For example, the composition may comprise from about 60 to about 95%, or about 60 to about 90%, or about 65 to about 90%, or about 65 to about 90% w/w based on the total weight of the composition. In various embodiments, the composition comprises the non-aqueous carrier in an amount of from about 30 to about 95%, or about 30 to about 90%, or about 35 to about 95%, or about 35 to about 90%, or about 40 to about 95%, or about 40 to about 90%, or about 45 to about 95%, or about 45 to about 90%, or about 50 to about 90%, or about 50 to about 90% w/w based on the total weight of the composition.

In some embodiments, the oral composition is non-aqueous. In some embodiments, the composition is substantially anhydrous. In some embodiments, the composition comprises less than 7% water, less than 5% water, less than 3% water, less than 2% water, less than 1% water, less than 0.5% water, less than 0.1% water, or less than 0.01% water, w/w based on the total weight of the composition. In the present composition, water includes absorbed moisture from the environment.

### Emulsifier

The oral composition may further comprise an emulsifier. Advantageously, the emulsifier may facilitate dispersion of the solid particles in the composition. For example, the emulsifier may facilitate dispersion of the probiotic in the composition. The emulsifier may be a non-ionic surfactant or an amphoteric surfactant. Examples of non-ionic surfactants include, but are not limited to polysorbate 80 (Tween 80), sorbitan oleate (Span 80), and polyoxyl 35 castor oil (Cremaphor EL). Examples of amphoteric surfactants include, but are not limited to, lecithin, such as egg lecithin and soybean lecithin, and phosphatidylcholine.

In some embodiments, the oral composition comprises the emulsifier in an amount of about 0.1 to about 5% w/w based on the total weight of the composition. For example, the composition may comprise the emulsifier in an amount of about 0.5 to about 4%, about 0.5 to about 3%, or about 0.5 to about 2% w/w based on the total weight of the composition. In various embodiments, the composition may comprise the emulsifier in an amount of about 1.5 to about 3.5% or about 2 to about 3% w/w based on the total weight of the composition. In some embodiments, the composition comprises the emulsifier (e.g. Tween 80, polysorbate 80) in an amount of about 1% w/w based on the total weight of the composition. In some embodiments, the composition comprises the emulsifier (e.g. Tween 80, polysorbate 80) in an amount of about 2.5% w/w based on the total weight of the composition.

### Abrasive

The oral composition may comprise an abrasive to improve the cleaning properties of the composition, e.g., for removing stains and/or plaque from teeth and/or polishing teeth. Suitable abrasives include, but are not limited to, silica, calcium carbonate, sodium bicarbonate, sodium chloride, sodium salts, phosphate salts, magnesium carbonate, hydrated aluminum oxides, clay (e.g. bentonite and kaolin), activated charcoal, tetrasodium pyrophosphate, disodium pyrophosphate, or a combination of any two or more thereof.

Those persons skilled in the art will appreciate that the amount of the abrasive may be selected to achieve the desired cleaning properties. Increasing the amount of the abrasive improves the cleaning properties of the composition. However, the inclusion of too much abrasive may cause the composition to damage teeth, e.g. the enamel of teeth, when the composition is used for oral cleaning. For example, the composition may comprise an abrasive in an amount of about 1 to about 40%, about 1 to about 30%, about 1 to about 25%, about 1 to about 20%, about 5 to about 40%, about 5 to about 30%, about 5 to about 25%, about 5 to about 15%, about 10 to about 35%, about 15 to about 30%, about 6 to about 14%, about 7 to about 13%, about 8 to about 12%, about 9 to about 11%, or about 10% w/w based on the total weight of the composition. In some embodiments, the composition comprises an abrasive in an amount of about 1%, about 2%, about 3%, about 4%, about 5%, about 6%, about 7%, about 8%, about 9%, about 10%, about 11%, about 12%, about 13%, about 14%, or about 15%, about 16%, about 17%, about 18%, about 19%, about 20%, about 21%, about 22%, about 23%, about 24%, about 25%, about 26%, about 27%, about 28%, about 29%, or about 30% w/w based on the total weight of the composition.

In some embodiments, the abrasive may also affect the viscosity of the composition, for example calcium carbonate, sodium bicarbonate, magnesium carbonate, hydrated aluminum oxides, clay (e.g. bentonite and kaolin), and activated charcoal.

The skilled person in the art will appreciate that in some embodiments, other components of the composition, such as the buffering agent, may also function as an abrasive, for example calcium carbonate, sodium chloride, phosphate salts, and sodium salts.

### Antibacterial Agent

Examples of antibacterial agents include, but are not limited to, xylitol, erythritol, antibacterial honey (such as Manuka and Kamahi honey), propolis, and tea tree oil. Preferably, the antibacterial agent is xylitol. Those persons skilled in the art will appreciate that some antibacterial agents may harm the probiotic. Accordingly, such antibacterial agents should be avoided or used in amounts that are low enough to avoid substantially reducing the amount of, or efficacy of, the probiotic in the oral composition.

The composition may comprise an antibacterial agent in an amount of about 1 to about 20%, about 2 to about 15%, about 3 to about 10%, about 4 to about 6%, or about 5% w/w based on the total weight of the composition. The composition may comprise an antibacterial agent in an amount of about 2 to about 10%, or about 2 to about 8% w/w based on the total weight of the composition. In some embodiments, the composition comprises an antibacterial agent in an amount of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% w/w based on the total weight of the composition. In some embodiments the composition comprises the antibacterial agent (e.g. xylitol) in an amount of about 5% w/w based on the total weight of the composition. In some embodiments the composition comprises the antibacterial agent (e.g. xylitol) in an amount of about 2% w/w based on the total weight of the composition.

### Fluoride Source

The oral composition may comprise a fluoride source, e.g., to improve the therapeutic properties of the composition. Advantageously, an oral composition comprising a fluoride source, when administered to an oral cavity, may strengthen tooth enamel and/or reduce dental caries. The fluoride source may be a fluoride salt. For example, the fluoride source may be sodium monofluorophosphate, sodium fluoride, stannous fluoride, or a combination of any two or more thereof.

The amount of the fluoride source may depend on commercial and/or regulatory factors, e.g. to comply with medical guidelines or regulatory requirements. In some embodiments, the composition comprises a fluoride source in an amount of about 0.38% to about 3.8%, or about 0.76% w/w based on the total weight of the composition. In some embodiments, the composition comprises a fluoride source in an amount of about 0.3 to about 4%, or about 0.4 to about 3%, or about 0.5 to about 2%, or about 0.6 to about 1%, or about 0.7% w/w based on the total weight of the composition. In some embodiments, the composition comprises a fluoride source in an amount of about 0.5 to about 1.5%, or about 0.6 to about 1.2% w/w based on the total weight of the composition.

### Sweetener

The oral composition may comprise a sweetener. Suitable sweeteners include, but are not limited to mogroside sweetener (monk fruit extract), sucralose, stevia, aspartame, saccharin, thaumatin, sorbitol, maltodextrin, isomalt, sucrose, honey, Synergy Powder Sweetener PF 7513, or a combination of any two or more thereof. Synergy Powder Sweetener PF 7513 contains 79% maltodextrin and 21% natural flavouring substances.

In some embodiments, the composition comprises a sweetener in an amount of about 0.05 to about 5%, about 0.1 to about 4%, about 0.2 to about 3%, about 0.3 to about 2%, about 0.4 to about 1%, or about 0.5% w/w based on the total weight of the composition. In some embodiments, the composition comprises a sweetener (e.g. Synergy Powder Sweetener) in an amount of about 0.5% w/w based on the total weight of the composition. In some embodiments, the composition comprises a sweetener (e.g. Synergy Powder Sweetener PF 7513) in an amount of about 3% w/w based on the total weight of the composition.

### Flavouring Agent

The composition may comprise a conventional flavouring agent as known in the art. Examples of flavouring agents include, but are not limited to, spearmint oil, peppermint oil, orange flavour, aniseed flavour, vanilla flavour, clove oil, lime flavour, Smoothenol Flavour 30712, and a combination of any two or more thereof. Smoothenol Flavour 30712 may comprise maltodextrin, gum arabic and triacetin and is known to bind to tongue receptors for masking bitter or metallic after taste. Those persons skilled in the art will appreciate that the flavouring agent selected must be compatible with probiotic viability.

The composition may comprise a flavouring agent(s) each in an amount of, e.g. about 0.01 to about 10%, about 0.01 to about 9%, about 0.01 to about 8%, about 0.01 to about 7%, about 0.01 to about 6%, about 0.01 to about 5%, about 0.5 to about 4.5%, about 1 to about 4%, about 1.5 to about 3.5%, about 2 to about 3%, or about 2.5% w/w based on the total weight of the composition. In some embodiments, the composition comprises a flavouring agent in an amount of about 0.1%, about 0.5%, about 1%, about 1.5%, about 2%, about 2.5%, about 3%, about 3.5%, about 4%, about 4.5%, or about 5% w/w based on the total weight of the composition. In some embodiments, the composition comprises a flavouring agent (e.g. spearmint flavouring oil) in an amount of about 2.5% w/w based on the total weight of the composition. In some embodiments, the composition comprises a flavouring agent (e.g. peppermint flavouring oil) in an amount of about 2.75% w/w based on the total weight of the composition. In some embodiments, the composition comprises a flavouring agent (e.g. Smoothenol Flavour 30712) in an amount of about 0.3% w/w based on the total weight of the composition.

In various embodiments, the composition comprises at least one flavouring agent. In some embodiments, when multiple flavouring agents are used, the composition comprises from about 0.1% to about 4% w/w of each flavouring agent, for example from about 0.5% to about 4%, or about 1% to about 4%, or about 1.5% to about 4%, or about 2% to about 4%, from about 0.5% to about 3%, or about 1% to about 3%, or about 1.5% to about 3%, or about 2% to about 3% w/w of each flavouring agent. In some embodiments, the composition comprises 2.5% w/w spearmint flavouring oil, 2.75% w/w peppermint flavouring oil, and 0.3% w/w Smoothenol Flavour 30712.

### Additional Additives

Those persons skilled in the art will appreciate the oral composition may comprise other additives conventionally used in an oral composition, such as a toothgel or toothpaste. Art skilled readers will further appreciate that additives need to be compatible with probiotic viability and efficacy. Such additives may provide or improve a therapeutic, cosmetic, stability, appearance and/or organoleptic property of the composition. Examples of suitable additives include, but are not limited to, a colourant (e.g. a food grade dye such as Brilliant blue or Food green, titanium dioxide, or white colouring), a foaming agent (e.g. polysorbate 80), a whitening agent (e.g. carbamide peroxide or hydrogen peroxide), a tooth sensitivity agent (e.g. potassium nitrate, arginine or stannous fluoride), an antioxidant (e.g. vitamin C or vitamin E), other anti-cariogenic agents (e.g. xylitol, fluoride, Manuka honey or tannins), a remineralisation agent (e.g. hydroxyapatite or calcium phosphate), prebiotics (e.g. galactose or raffinose), and/or natural extracts (e.g. amla (Phyllanthus emblica), neem (Azadirachta indica), clove (Syzygium aromaticum), tulsi (Ocimum tenuiflorum), or turmeric (Curcuma longa)). Such additives may be included in the oral composition of the invention in amounts typical for oral formulations. A variety of pharmaceutically acceptable additives suitable for oral administration of viable or lyophilized bacteria are well known in the art (see, for example, Remington's Pharmaceutical Sciences, 18th ed., Gennaro, ed., 1990, Mack Publishing Co., Easton, Pa., or Remington's Pharmaceutical Sciences, 23^{rd} ed., Adejare, ed., 2021, Academic Press Inc., incorporated herein by reference).

The applicants have surprisingly found that many common foaming agents kill or are inhibitory for M18 and K12. It is not possible to add BLIS M18 and/or K12 to a commercial formulation because the microorganisms are killed because of standard toothpaste components such as glycerol, foaming agents, and/or water.

### Form of the Oral Composition

The oral composition may be in the form of a gel or a paste, e.g., a toothpaste. The viscosity of the composition may be greater than 1,900,000 cp at 25° C. The viscosity of the composition may be from about 20,000 to about 2,000,000 cp at 25° C. The viscosity of the composition may be from about 20,000 to about 500,000 cp at 25° C., for example from about 30,000 to about 400,000, or about 40,000 to about 100,000, or about 40,000 to about 70,000 cp at 25° C. When the composition is in the form of a gel, the viscosity of the composition is in some embodiments between about 35,000 to about 65,000 cp at 25° C. When the composition is in the form of a paste, the viscosity of the composition is in some embodiments between about 70,000 to 100,000 cp at 25° C.

Viscosity may be measured at 25° C. using a Brookfield LVDVI Prime using Brookfield Helipath Spindle (S96) set at 0.3 RPM. A skilled worker will appreciate other methods that can be used to measure viscosity.

Apart from chemical stability and release characteristics, physical stability of the composition in terms of “syneresis” is another challenge encountered commonly with oil-based formulations. Syneresis is the leakage of oil from the composition due to contraction of the gel network upon storage. To overcome syneresis, the amount of the viscosity modifier and/or buffering agent may be increased to reduce syneresis. However, addition of too much of these components may result in highly viscous systems (semisolid). If the viscosity of the composition is too high, it may be difficult to dispense as a toothpaste, or prevent or inhibit the release of the probiotic. Preferably, the gel network is strong enough to maintain physical stability upon storage, but weak enough to allow for the release of the probiotic with agitation such as brushing teeth or spreading within the oral cavity. Preferably, the gel network is tough enough to handle temperature and humidity and physical stresses upon storage to maintain the gel structure, but soft enough to break open upon agitation such as brushing teeth or due to salivary flow. Gel properties such as gel strength, bloom strength, bio-adhesion, and consistency may be measured using a texture analyser.

Syneresis may be measured using accelerated syneresis or real time syneresis. Accelerated syneresis can be measured using the centrifugal acceleration test described in Journal of Dairy Science, Vol: 100, Issue: 2, Page: 901-907. Briefly, 1 g of the formulation is weighed in a separate 1.5 mL Eppendorf tube and centrifuged (Eppendorf centrifuge 5415D, Lab supply NZ) at 13000 rpm for 1, 5, and 10 minutes at room temperature. The volume of liquid leaked (supernatant) at each time point is transferred into another Eppendorf tube and weighed. The cumulative percentage of the leaked liquid is calculated and the syneresis compared. Real time syneresis is measured using the same procedure, except the sample is not subjected to centrifugal acceleration but any spontaneous leak upon storage due to contraction of the formulation is collected and measured.

In some embodiments, the composition has a syneresis ratio of about 10% to about 40% after being centrifuged at 13,000 rpm for 5 minutes at 25° C. In some embodiments, the oral composition has a syneresis ratio of about 20% to about 40% after being centrifuged at 13,000 rpm for 5 minutes at 25° C. In some embodiments, the oral composition has a syneresis ratio of about 30% after being centrifuged at 13,000 rpm for 5 minutes at 25° C. In some embodiments, the oral composition has a syneresis ratio of about 20% to about 40% after being centrifuged at 13,000 rpm for 10 minutes at 25° C. In some embodiments, the composition has a syneresis ratio of about 10% to about 20%, or about 13% to about 18% after being centrifuged at 13,000 rpm for 5 minutes at 25° C. In some embodiments, the oral composition has a syneresis ratio of about 30% after being centrifuged at 13,000 rpm for 10 minutes at 25° C. In various embodiments, syneresis may be measured at 22° C.

For efficacy in the oral environment, the composition of the invention needs to be carefully formulated to provide Streptococcus salivarius at amounts over time which facilitate colonisation of the oral cavity of the subject treated. Advantageously, the composition needs to be formulated to avoid immediate release of all the Streptococcus salivarius probiotic, and provide sustained release of Streptococcus salivarius over time.

Release of the Streptococcus salivarius from the composition is described by cumulative percentage release of the total Streptococcus salivarius in the composition, e.g. 35% of the total Streptococcus salivarius in the composition.

In some embodiments, release of the Streptococcus salivarius from the composition at 15 minutes after being administered to an oral cavity is in the range of from about 35% to about 95%. In some embodiments, release of the Streptococcus salivarius from the composition at 15 minutes after being administered to an oral cavity is in the range of from about 40% to about 95%. In some embodiments, release of the Streptococcus salivarius from the composition at 15 minutes after being administered to an oral cavity is in the range of from about 35% to about 90%. In some embodiments, release of the Streptococcus salivarius from the composition at 15 minutes after being administered to an oral cavity is in the range of from about 40% to about 90%. In some embodiments, release of the Streptococcus salivarius from the composition at 15 minutes after being administered to an oral cavity is in the range of from about 40% to about 85%, or from about 40% to about 80%, or from about 40% to about 75%.

In some embodiments, release of the Streptococcus salivarius from the composition at 30 minutes after being administered to an oral cavity is in the range of from about 50% to about 98%. In some embodiments, release of the Streptococcus salivarius from the composition at 30 minutes after being administered to an oral cavity is in the range of from about 50% to about 95%. In some embodiments, release of the Streptococcus salivarius from the composition at 30 minutes after being administered to an oral cavity is in the range of from about 55% to about 98%. In some embodiments, release of the Streptococcus salivarius from the composition at 30 minutes after being administered to an oral cavity is in the range of from about 55% to about 95%. In some embodiments, release of the Streptococcus salivarius from the composition at 30 minutes after being administered to an oral cavity is in the range of from about 60% to about 98%, or from about 60% to about 95%, or from about 60% to about 93%.

In some embodiments, the composition releases about 40% of the Streptococcus salivarius at about 15 minutes after being administered to the oral cavity. In some embodiments, the composition releases about 60% of the Streptococcus salivarius at about 30 minutes after being administered to the oral cavity.

In some embodiments, release of the Streptococcus salivarius from the composition at 60 minutes after being administered to an oral cavity is in the range of from about 60% to about 100%. In some embodiments, release of the Streptococcus salivarius from the composition at 60 minutes after being administered to an oral cavity is in the range of from about 65% to about 100%.

Without wishing to be bound by theory, it is believed that the release of the probiotic from the formulation indicates the likely success of the probiotic in terms of its ability to colonise. If release from the formulation is too fast, then it may have a low colonisation success profile. Most probiotic will be quickly swallowed down the throat with the saliva. Conversely, if the release from the formulation is too slow then the probiotic may not get to levels high enough to allow colonisation of the oral cavity. Therefore, a finely tuned release profile is desired that allows the appropriate release and colonisation to occur.

### Method of Preparing an Oral Composition

The oral composition described herein may be prepared by first mixing a non-aqueous carrier and an emulsifier to provide a mixture. Streptococcus salivarius, a buffering agent, and optionally other solid components, are added and dispersed throughout the mixture using simple mixing. A viscosity modifier is then added to the mixture, and the mixture is homogenised, e.g., for about 3 to about 5 minutes to provide the oral composition. In some embodiments, the mixture is homogenised with a high shear homogeniser or an overhead stirrer.

Alternatively, the oral composition may be prepared by a method comprising heating a non-aqueous carrier, e.g. sunflower oil, to a temperature between about 140° C. to about 150° C. Next, a viscosity modifier is added to the heated non-aqueous carrier and the mixture is stirred, e.g., at a rate of about 600 to about 700 rpm. In some embodiments, the mixture is stirred until the viscosity modifier, e.g., ethyl cellulose, is solubilised to provide a clear mixture. The mixture is then allowed to partially cool, e.g., to a temperature of about 80° C. to about 90° C. followed by addition of an emulsifier to the mixture. The mixture is allowed to further cool, e.g., to about 25° C. to about 35° C., then a Streptococcus salivarius is added. The mixture is then homogenised to provide the oral composition. In some embodiments, the mixture is homogenised with a high shear homogeniser or an overhead stirrer.

### Method of Improving Oral Health

The composition is useful for improving the oral health of a subject. For example by preventing or treating any of the conditions identified in WO2001027143, WO2002070719, and WO2005007178 (supra), and all incorporated herein by reference in their entireties. Streptococcus salivarius M18 is also known to help reduce dental plaque, support oral health and oral flora, reduce dental caries, prevent dental caries, treat and prevent gingivitis, and treat and prevent periodontitis (Burton, J. P., et al., 2013 J. Med. Microbiol. 62, 875-884; Burton, J. P., et al., 2013, PLoS ONE 8.; Di Pierro, et al. 2015. Clin Cosmet Investig Dent. 7:107-13; L Scariya, D. V, N., M Varghese, 2015. Int. J. Pharma Bio Sci. 6, 242-250).

Accordingly, disclosed herein is a method of improving the oral health of a subject, comprising administering an oral composition as described herein to the oral cavity of the subject. In some embodiments, the method is for reducing dental caries in a subject. In some embodiments, the method is for removing and/or preventing stains and/or plaque on the teeth of the subject. In some embodiments, the method is for strengthening the enamel on the teeth of the subject. In some embodiments, the method is for treating and/or preventing gingivitis. In some embodiments, the method is for assisting gum healing. In some embodiments, the method is for preventing halitosis.

Also disclosed herein is a method of delivering a probiotic to the oral cavity of a subject, comprising administering an oral composition as described herein to the oral cavity of the subject. Advantageously, the probiotic may at least partially colonise the oral cavity. Preferably, the probiotic is a Streptococcus salivarius, such as M18, K12, or a combination thereof.

In some embodiments, administering the oral composition to the oral cavity of the subject comprises administering the composition to the teeth, gums, tongue, buccal cavity, and/or periodontal pocket of the subject. For example, the composition may be administered to the oral cavity by brushing, spotting, coating, massaging on the teeth, gums, tongue, and/or buccal cavity of the subject, or by filling a periodontal pocket of the subject. Alternatively, the method of improving the oral health of a subject may comprise administering the oral composition as described herein to a denture or mouthguard. The composition may be administered to the denture or mouthguard, wherein the denture or mouthguard is within the oral cavity of the subject or external to the oral cavity. The composition may be useful for, but is not limited to use in, pre-dentate children or xerostomia patients.

In some embodiments, the composition is expectorated after administration to the oral cavity, e.g., the composition may be expectorated immediately after being administered to the oral cavity. In some embodiments, the composition is retained in the oral cavity for at least about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 25 minutes, or about 30 minutes. In some embodiments, the composition is not expectorated after administration to the oral cavity.

In some embodiments, the subject is a human. In some other embodiments, the subject is an animal, such as a dog, cat, horse, sheep, cow, or other domestic or farm animal.

The following non-limiting examples are provided to illustrate the present invention and in no way limit the scope thereof.

### EXAMPLES

### Method of Preparing Toothpaste (Formulations A4, A5, A6, and A-7, Table 1)

The non-aqueous carrier and emulsifier (Tween 80) were mixed gently in a beaker until the liquid became cloudy. Streptococcus salivarius M18, Streptococcus salivarius K12 (for A-7), calcium carbonate, and xylitol were added to the beaker and gently mixed to disperse the solid in the liquid medium. Hydrophobic silica was then added, and the mixture was homogenised for 3-5 minutes (intermittently to avoid heat build-up) using a high shear homogeniser (Ultra Turrax) or air overhead stirrer.

### Method of Preparing Silica Toothgel (Formulation A3, Table 1)

The non-aqueous carrier (olive oil) and emulsifier (Tween 80) were mixed gently in a beaker until the liquid became cloudy. Streptococcus salivarius M18 was added to the beaker added and gently mixed to disperse the solid in the liquid medium. Hydrophobic silica was then added, and the mixture was homogenised for 3-5 minutes (intermittently to avoid heat build-up) using a high shear homogeniser (Ultra Turrax) or air overhead stirrer.

### Method of Preparing Ethyl Cellulose Toothgel (Formulation A1 and A2, Table 1)

The non-aqueous carrier (Sunflower oil) was heated on a magnetic stirrer hotplate to a temperature of 140-160° C. Ethyl cellulose was added slowly to the hot oil with continuous stirring (600-700 rpm) using magnetic stirrer bar. The rpm used to achieve good solubilisation of the ethyl cellulose were between 600 and 700 rpm. Higher speeds may form bubbles and a lower speed may not be enough to completely solubilise the ethyl cellulose. Slow addition and continuous stirring are preferable to avoid the formation of ethyl cellulose lumps. Once the ethyl cellulose is completely dissolved to provide a clear solution (about 40 minutes), heating was stopped, and the beaker was moved to a non-heated stirrer plate. The mixture was allowed to cool down slowly with stirring. Once the temperature of the mixture reached 80-90° C., the emulsifier (Tween 80 or Span 80) was added followed by beeswax with continuous stirring until a homogeneous mixture was achieved. The mixture was allowed to cool down to room temperature (about 25° C.-35° C.). Streptococcus salivarius M18, and other solids such as xylitol and sweetener, were added with continuous stirring. A high shear homogeniser or air overhead blender may be used in some instances to improve the homogeneity of the mixture.

### Example 1: Example Formulations

Examples of toothgel (A-1 to A-3) and toothpaste (A-4 to A-7) formulations prepared according to the aforementioned methods are shown in Table 1. Also shown in Table 1 are comparative formulations C-1 to C-3. C-1 was prepared according to example 1 in WO2017195074A1. C-2 was prepared according to example 3 in WO2012097429A1. C-3 was prepared according to example 7 in WO2010054439A1, except without application to beads. All amounts are shown in % w/w based on the total weight of the formulation.

TABLE 1

| | A-1 | A-2 | A-3 | A-4 | A-5 | A-6 | A-7 | C-1 | C-2 | C-3 |
|---|---|---|---|---|---|---|---|---|---|---|
| | % w/w | % w/w | % w/w | % w/w | % w/w | % w/w | % w/w | % w/w | % w/w | % w/w |
| Formulation | (cfu/g) | (cfu/g) | (cfu/g) | (cfu/g) | (cfu/g) | (cfu/g) | (cfu/g) | (cfu/g) | (cfu/g) | (cfu/g) |
| S. salivarius M18 | 6 | 6 | 6 | 1.05 | 1.05 | 1.05 | 2.5 | 1 | 1.2 | 40 |
| | (4.3 × | (3.3 × | (3.9 × | (3.1 × | (3.1 × | (3.1 × | (5.43 × | (1.27 × | (2.62 × | (7.8 × |
| | 10⁹ cfu/g) | 10⁹ cfu/g) | 10⁹ cfu/g) | 10⁸ cfu/g) | 10⁸ cfu/g) | 10⁸ cfu/g) | 10⁹ cfu/g) | 10⁹ cfu/g) | 10⁻⁹ cfu/g) | 10¹⁰ cfu/g) |
| S. salivarius K12 | — | — | — | — | — | — | 2.9 | — | — | — |
| | | | | | | | 6.3 × 10⁹ | | | |
| | | | | | | | cfu/g | | | |
| Hydrophobic silica | — | — | 4 | 6 | 5 | 5 | 6 | — | — | — |
| Ethyl cellulose EC-100 | 4 | 4 | — | — | — | — | — | — | — | — |
| Bees wax | 2 | 2 | — | — | — | — | — | — | 12.7 | — |
| Polysorbate 80 | — | — | 1 | 1 | 1 | 1 | 1 | — | 6.4 | — |
| Span 80 | 1 | 1 | — | — | — | — | — | — | — | — |
| Calcium carbonate | — | — | — | 15 | 15 | 25 | — | — | — | 5 |
| Baking soda | — | — | — | — | 10 | — | — | — | — | — |
| Fluoride (sodium | — | — | — | — | — | 0.76 | — | — | — | — |
| monofluorophosphate) | | | | | | | | | | |
| Xylitol (fine grain) | — | 10 | — | 5 | 5 | 5 | — | 17.6 | — | — |
| Tapioca starch | — | — | — | — | — | — | — | — | 14.8 | — |
| Monobasic potassium | — | — | — | — | — | — | — | 0.19 | — | — |
| phosphate | | | | | | | | | | |
| dibasic potassium | — | — | — | — | — | — | — | 1.32 | — | — |
| phosphate | | | | | | | | | | |
| Synergy Powder | — | — | — | 0.5 | 0.5 | 0.5 | — | — | 0.2 | — |
| Sweetener (PF 7513) | | | | | | | | | | |
| Flavouring agent | — | — | — | 2.5 | 2.5 | 2.5 | | 0.11 | 4.9 | — |
| Canola oil | — | — | — | — | — | — | | 4.3 | — | 55 |
| Blend of hydrogenated | — | — | — | — | — | — | | 75.5 | — | — |
| oils and emulsifiers | | | | | | | | | | |
| (coconut oil + soy | | | | | | | | | | |
| lecithin) | | | | | | | | | | |
| Caprylic/Capric | — | — | — | 71.95 | 59.95 | 59.19 | 88.00 | — | — | — |
| Triglyceride (MCT) | | | | | | | | | | |
| Sunflower oil | — | — | — | — | — | — | | — | 61.1 | — |
| Olive oil | 87 | 87 | 89 | — | — | — | | — | — | — |

TABLE 2
Suppliers

| | |
|---|---|
| S. salivarius M18 | BLIS Technologies (NZ) |
| S. salivarius K12 | BLIS Technologies (NZ) |
| Hydrophobic silica (Aerosil R972) | Evonik, Germany |
| | (supplied by Chemiplas NZ) |
| Ethyl cellulose EC-100 | (ETHOCEL ™ M |
| | Premium Standard 100 cp |
| | Polymer) obtained from |
| | Colorcon, Australia) |
| White beeswax | Henry Lamotte (Germany) |
| Polysorbate 80 (Tween 80) | Pure Ingredients NZ/Sigma |
| | Aldrich NZ (Pharma grade) |
| Span 80 | Sigma NZ (pharma grade) |
| Calcium carbonate | Pure Ingredients NZ/ |
| | Pan Reac-AppliChem |
| | (ITW reagents, NZ) |
| Xylitol (fine grain) | Roquette, Germany |
| Tapioca starch | Pams NZ |
| Monobasic potassium phosphate | Sigma Aldrich NZ |
| dibasic potassium phosphate | Sigma Aldrich NZ |
| Synergy Powder Sweetener (PF 7513) | Pacific Flavours NZ |
| Canola oil | Pams, NZ |
| Blend of hydrogenated oils and | Kremelta vegetable |
| emulsifiers (coconut oil + soy lecithin) | shortening, NZ |
| Caprylic/Capric Triglyceride (MCT) | Miglyol 812 - Sasol |
| | Germany (Pharma grade) |
| | Radia 7104, Oleon, Malaysia |
| Sunflower oil | Pams NZ or Sigma |
| | NZ (Pharma grade) |
| Olive oil | Pams NZ or Lipoid, |
| | Germany (Pharma grade) |
| Baking soda (Sodium bicarbonate) | Redox NZ |
| Fluoride (sodium | Alfa Aesar, Thermo |
| monofluorophosphate | Fisher Scientific NZ |
| Spearmint flavouring oil | Pure Ingredients NZ, |

### Appearance and Acceptability of the Formulations

The formulations were assessed for palability by assessing the visual, olfactory and taste sensory properties of the formulations.

A-4 had an opaque to white appearance with desirable paste like consistency and flow properties.

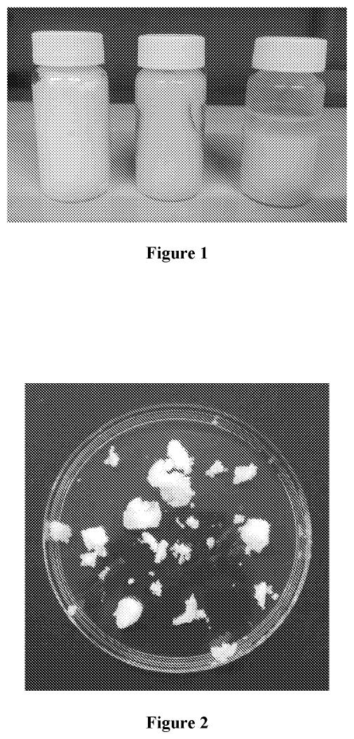

Formulations C-1 to C-3 are shown in FIG. 1. C-1 was similar to A-4 at the time of manufacturing, but upon storage turned to brittle flakes of unpleasant appearance (see FIG. 2). Due to the rock-like and flaky form of the composition, C-1 was not in a suitable form to be used as a toothpaste or gel. C-2 had a smooth waxy, yellowish appearance but turned to solid rock like when kept at 5° C. C-3 was a thick suspension with poor flow property. This form was not suitable for use as a toothpaste.

### Stability Testing

The stability of the formulations was assessed by measuring the amount of the probiotic that remains viable upon storage.

The formulations were packaged into plastic tubes with very low water vapour transfer rate or glass vials. The formulations were then placed into an incubator at 25° C. and 60% relative humidity (RH) or at 5° C. Probiotic viability was measured through standard enumeration methods at designated time points.

After 6 months, formulations A-4 and A-6 overall exhibited good stability at both 25° C./60% RH and 5° C. in both glass vials and toothpaste tubes. A-4 and A-6 maintained a constant viable cell count of the probiotic at both temperatures.

A-1 to A-3 exhibited good stability at 25° C./60% RH in glass vials for at least 30 months.

### Syneresis

Syneresis was measured in formulations A-4 and C-1 to C-3. Two forms of syneresis were measured: accelerated and real time.

The accelerated syneresis study involved subjecting the formulations to centrifugation at 13,000 rpm for 1, 5 and 10 minutes. The amount of syneresis in each formulation was then measured.

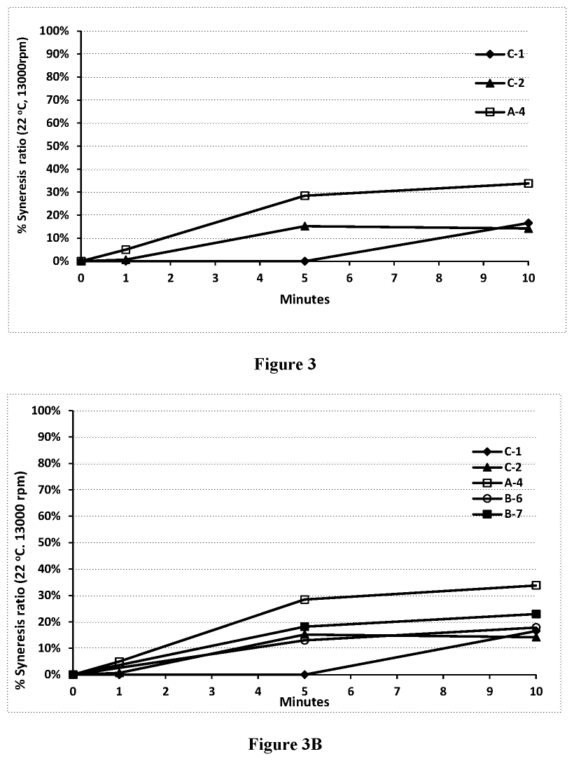

The results of the accelerated syneresis study are shown in FIG. 3. A-4 showed syneresis at 1 minute and 5 minutes, and plateaued with minimal increase in syneresis after 10 minutes. Of the formulations tested, C-1 showed the least syneresis. C-1 did not show any syneresis until 10 minutes of centrifugation. Without wishing to be bound by theory, the low syneresis may suggest that the gel network is too strong. The strong gel network may prevent the release of the probiotic from the gel matrix. C-2 showed less syneresis compared to A-4. Like C-1, the low syneresis may be beneficial for the physical stability but may prevent release of the probiotic from the gel network. C-3 showed separation of the oil (supernatant) from the probiotic powder (sediment) immediately at 1 minute and the effect increased over the testing time. Since C-3 is not a gel, this effect is not syneresis but separation of liquid and solid phase.

In the real time syneresis study, the formulations were kept at 25° C. and 60% RH, and observed for syneresis.

No syneresis was observed for A-1, C-1 or C-2. As observed for the accelerated syneresis study, C-3 showed separation of the oil (supernatant) from the probiotic powder (sediment) after 1 month time point. Since C-3 is not a gel, this effect is not syneresis but separation of liquid and solid phase (FIG. 1).

### Release Profile

The release profiles of formulations A-1 to A-4, A-7 and C-1 to C-3 were assessed. BLIS M18 lozenges were used as a control. The formulations were placed into a release medium, i.e. a liquid reservoir to collect bacteria that escapes the formulation. The release medium was set to 37° C. and pH 6.7 to mimic the pH of saliva/oral cavity. The release medium was stirred at about 200 rpm using a magnetic stirrer bar to mimic salivary flow. 0.895 g of BLIS M18 lozenge or 0.5 g probiotic formulation were added (˜1.5×10⁹ cfu/dose) to the release medium. Samples were removed at T=0, 1, 5, 10, 15, 30, 60, 120 or 180 minutes by collecting a 100 μL aliquot of the release media, which was diluted in PBS (900 μL) and stored in −20° C. until enumerated. 100 μL of fresh release media at 37° C. was added to the beaker to maintain the release medium volume. At the time of analysis, samples from the lozenge or paste experiment were thawed to room temperature, and 100 μL of the solution was serially diluted to 10′ then spot plated on CAB K12 agar plate using 3×10 μL at each dilution. The plates were then placed in incubator a 37° C. and 5% CO₂ for 18-20 hours followed by enumeration manually or using automated colony counter.

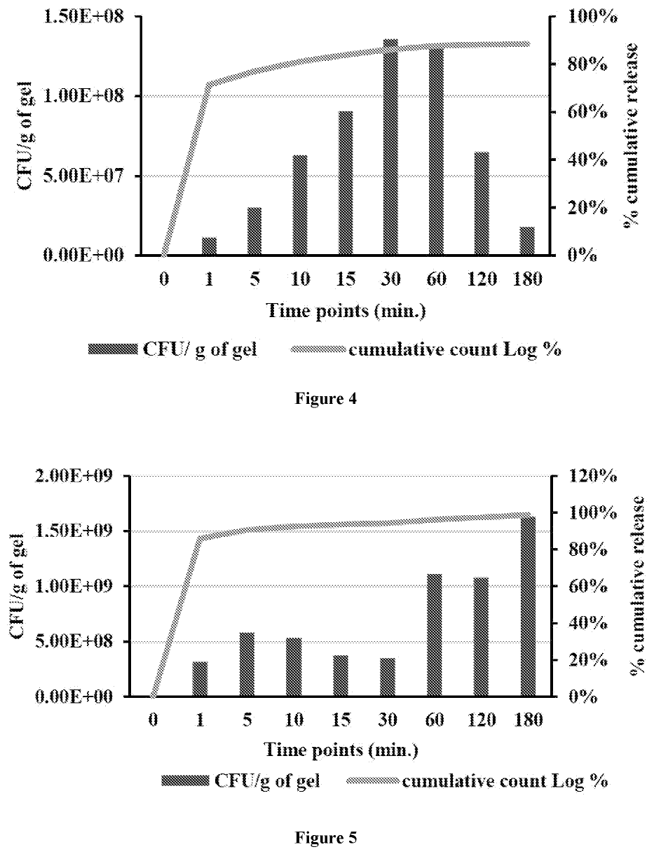

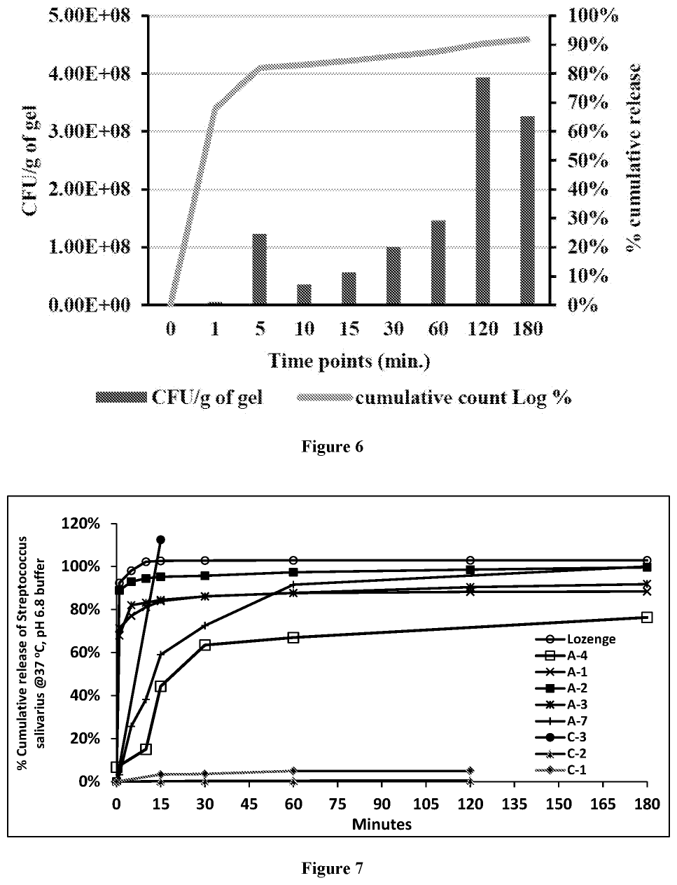

Results of the release profile experiments are shown in FIGS. 4-7. The BLIS M18 in a lozenge reached 100% by 10 minutes in this model. Formulations A-1 and A-3 showed delayed release with ˜85% in 30 min. A-2 showed a 95% release within 15 min. The cumulative release of probiotic for A-4 was about 45% in first 15 minutes with sustained release thereafter. These results indicate that the release of the probiotic from the paste or gel platforms may be regulated.

C-1 and C-2 did not effectively release probiotic (<5%) by 120 minutes. C-3 released its probiotic to 100% by 15 minutes. It is expected that due to the liquid nature of the formulations, instantaneous (100%) release of M18 would take place within 5 minutes.

### Colonisation

Studies were performed to investigate the ability of the probiotic formulations to colonise and increase the measurable levels of the probiotic within the oral cavity following BLIS lozenge or toothpaste containing Streptococcus salivarius M18.

A comparative trial was conducted in healthy adult human volunteers, where a BLIS lozenge containing Streptococcus salivarius M18 and Streptococcus salivarius K12 was administered twice daily for 7 days. Saliva samples collected 8 hours after the first dose and 24 hours after the last dose were analysed for Streptococcus salivarius M18 like colonies.

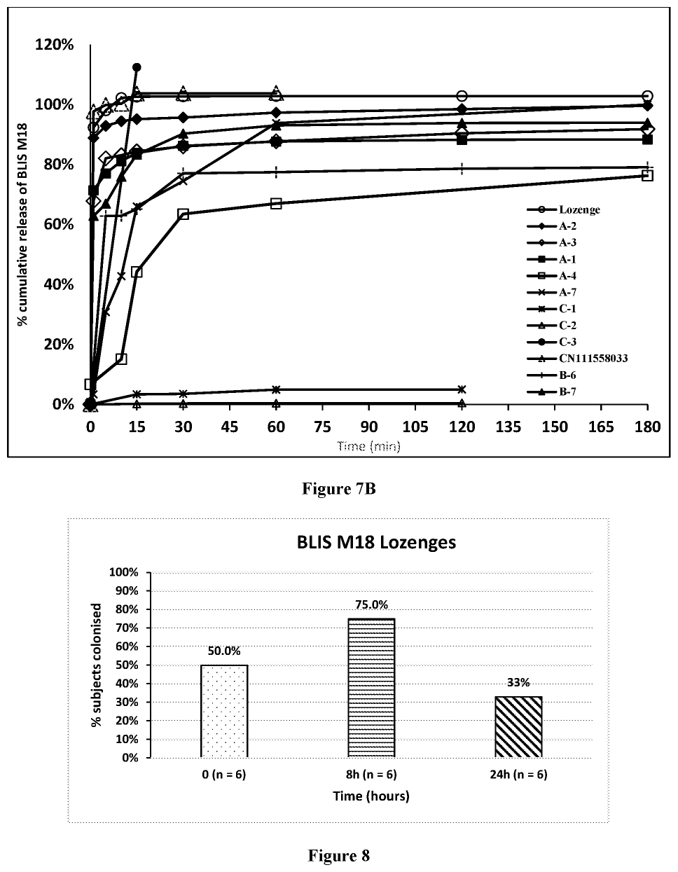

The lozenges demonstrated an increase in detectable probiotic in the saliva at 8 hours (see FIG. 8).

A similar protocol was used to study the colonisation properties of the A-4 formulation. Healthy human volunteers brushed their teeth with formulation A-4 twice daily for 7 days. Saliva samples were collected 8 hours after first dose and 24 hours after last dose.

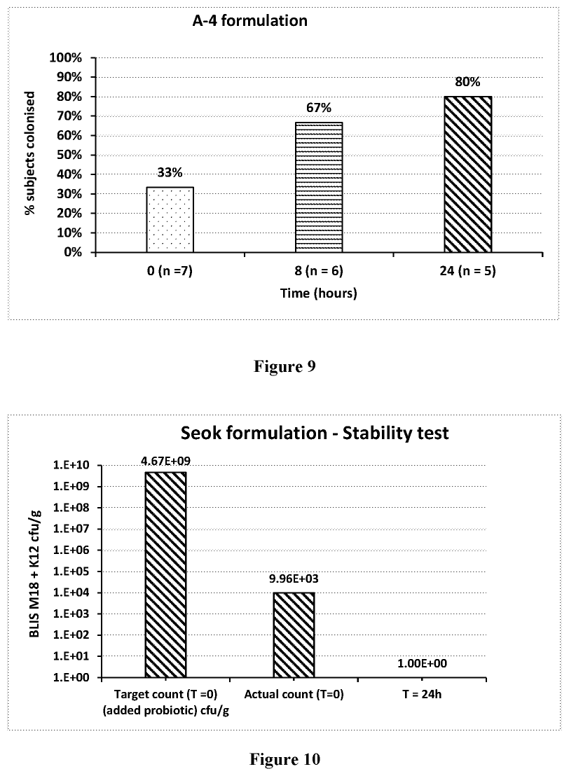

The data shows that similar colonisation can be achieved following 8 hours and better following 24 hours of the toothpaste formulation compared to the Streptococcus salivarius M18 containing lozenges (see FIG. 9). This is surprising as the toothpaste had comparable efficacy in colonisation to the lozenge formulation, despite being in the oral cavity for a shorter period of time.

### Example 2: Sensory Trial

Examples of toothpaste (B-1 to B-13) formulations prepared according to the aforementioned methods are shown in Table 3. All amounts are shown in % w/w based on the total weight of the formulation.

TABLE 3

| Formulation | | | | | | | | | | | | | |
|---|---|---|---|---|---|---|---|---|---|---|---|---|---|
| (% w/w) | B-1 | B-2 | B-3 | B-4 | B-5 | B-6 | B7 | B-8 | B-9 | B-10 | B-11 | B-12 | B-13 |
| S. salivarius | 1.23 | 1.23 | 1.23 | 1.23 | 1.23 | 1.23 | 1.23 | 1.23 | 1.23 | 1.23 | 1.23 | 1.23 | 1.23 |
| M18 (2 × 10⁹ | | | | | | | | | | | | | |
| CFU/g) | | | | | | | | | | | | | |
| Hydrophobic | 6 | 10 | 8 | 8 | 8 | 8 | 7 | 6 | 5 | 5 | 6 | 8 | 8 |
| silica | | | | | | | | | | | | | |
| Xanthan Gum | — | — | 5 | 5 | 5 | 5 | 2 | — | — | — | — | — | 5 |
| Fluoride | — | — | 0.76 | 0.76 | 0.76 | 0.76 | 0.76 | — | 0.76 | 0.76 | — | — | 0.76 |
| (Na₂PFO₃) | | | | | | | | | | | | | |
| Smoothenol | — | — | — | 0.3 | 0.3 | 0.3 | 0.3 | — | — | — | — | — | — |
| Polysorbate 80 | 1 | 1 | 3 | 2.5 | 2.5 | 2.5 | 2.5 | 1 | 1 | 1 | 1 | 1 | 3 |
| Calcium | 25 | n/a | 20 | 20 | 20 | 20 | 20 | 15 | 25 | 25 | 25 | 25 | 25 |
| carbonate | | | | | | | | | | | | | |
| Xylitol | 5 | 15 | 5 | 5 | 5 | 5 | 2 | 5 | 5 | 5 | 5 | 5 | 5 |
| Spearmint | 2.5 | 2.5 | 2.5 | 2.5 | 2.5 | 2.5 | 2.5 | — | — | 2.5 | 2.5 | 2.5 | 2.5 |
| Flavouring Oil | | | | | | | | | | | | | |
| Peppermint oil | 2 | 2 | 2.5 | 2.5 | 3 | 2.75 | 2.75 | — | — | 1 | — | — | 2 |
| Synergy Powder | 0.5 | 0.5 | 1 | 3 | 3 | 3 | 3 | — | 0.50 | 0.50 | 0.50 | 0.50 | 0.50 |
| Sweetener | | | | | | | | | | | | | |
| White colour | — | 0.05 | — | — | — | — | — | — | — | — | — | — | — |
| Caprylic/Capric | 56.77 | 67.72 | 51.01 | 49.21 | 48.71 | 48.96 | 55.96 | 71.30 | 61.60 | 58.01 | 58.77 | 58.77 | 46.01 |
| Triglyceride | | | | | | | | | | | | | |
| (MCT) | | | | | | | | | | | | | |

TABLE 4
Additional suppliers

| | |
|---|---|
| Peppermint oil | Pure Nature, New Zealand |
| Xanthan gum | Lotus, New Zealand |
| Smoothenol | Sensient, New Zealand |
| White colour | Colour mill, New Zealand |

Sensory trials were carried out by getting 5-10 participants to use each toothpaste twice daily for 3 days and comparing to regular toothpaste. Participants rated a number of parameters including:
overall likeability
amount of foam
mouth freshness
ability to clean
thickness
like mouthfeel
like flavour
flavour strength
bitter/metallic after taste
prolonged aftertaste
pleasant to taste.

B3 was preferred compared to B1 and B2. B6 was preferred compared to B5 and B4. B6 and B7 were the most preferred formulations.

Compared to regular toothpaste, B3 and B7 were preferred overall and specifically in terms of mouth freshness and ability to clean.

Stability testing was carried out using the procedure described in Example 1. Samples B3, B6, B8, B9, and B10 were all found to be stable for at least 6 months at 25° C./60% RH (FIG. 12).

### Example 3: Comparative Study to Seok et al.

A formulation was prepared according to Seok, Y., & Lee, J. (2018). Formulating a probiotic toothpaste for vitamin B6 delivery system. Journal of International Research in Medical and Pharmaceutical Sciences, 13(2), 53-67.

TABLE 5
Composition according to Seok et al.

| Ingredient | % w/w | Vol (ml or g) | |
|---|---|---|---|
| BLIS K12 + M18 | 2.47 | 4 | g |
| Vitamin B6 | 0.62 | 1 | g |
| Salts (phosphate) | 3.09 | 5 | g |
| Glycerine (glycerol) | 6.17 | 10 | ml |
| Xanthan gum (pure nature) | 2.47 | 4 | g |
| Tetrasodium pyrophosphate* | 3.09 | 5 | g |
| Sodium bicarbonate | 12.35 | 20 | g |
| Sodium dodecyl sulphate | 1.54 | 2.5 | g |
| Hydrogen peroxide | 6.17 | 10 | ml |
| Emulsion medium** | 24.69 | 40 | g |
| Water | 37.04 | 60 | ml |

Replacements
*Calcium carbonate (Pure nature, New Zealand)
**10 ml Olive oil + 15 ml Tween 80 + 22.5 g Emulsifying wax

Azone not used

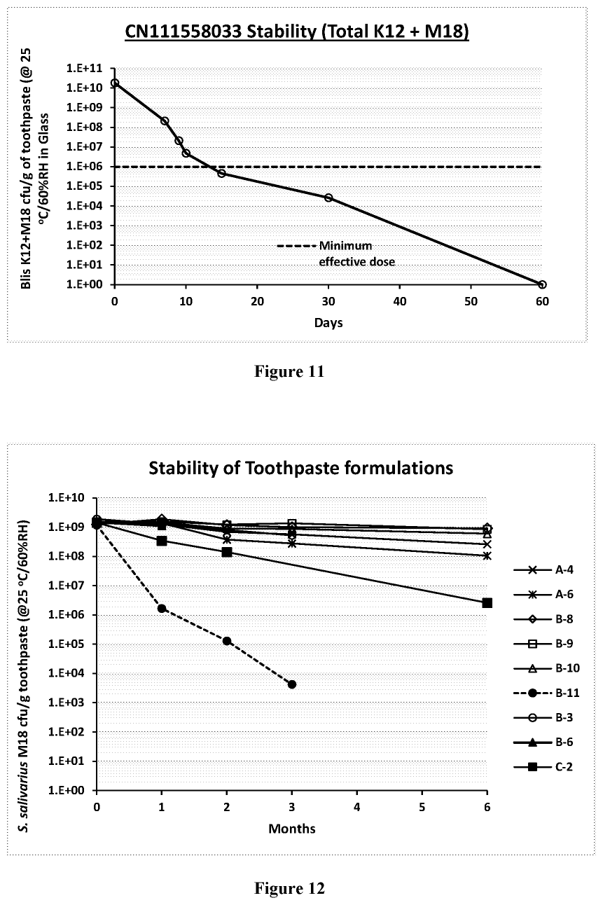

Stability testing was carried out according to Example 1. The cell count in the formulation of Seok dropped by a 6 log immediately. 24 hours after storage, there were no live BLIS M18 or K12 cells detectable (FIG. 10). It is expected that the cells were not viable due to the presence of water as a vehicle and antibacterial components sodium dodecyl sulphate, glycerine, azone and hydrogen peroxide.

### Example 4: Comparative Study to CN111558033

A formulation was prepared according to CN111558033.

TABLE 6
Composition according to CN111558033

| Ingredient | % w/w | Batch 100 g | |
|---|---|---|---|
| BLIS K12 | 7 | 7 | g |
| BLIS M18 | 7 | 7 | g |
| Calcium carbonate | 45 | 45 | g |
| Glycerol | 30 | 30 | ml |
| Sodium benzoate | 0.1 | 0.1 | g |
| Silica | 1 | 1 | g |
| Sodium lauroamphodiacetate* | 1 | 1 | ml |
| Sodium saccharin | 0.1 | 0.1 | g |
| Refined water | 8.8 | 8.8 | ml |

Replacements
*Tween 80

Stability testing was carried out according to Example 1. The cell count of the CN111558033 formulation dropped steadily over 60 days

Release profile testing was assessed using the method described in Example 1. The formulation of CN111558033 was found to release 98% of the Streptococcus salivarius in the composition within 1 minute, i.e. instantaneous release. It is expected that the Streptococcus salivarius was released instantaneously because the probiotic is suspended in a water vehicle and, therefore, instantaneously dispersed in the release medium.

### Example 5: Comparison of Foaming Agents

This example was performed to demonstrate the effect of different foaming agents on the viability of BLIS M18.

Samples were prepared by
1. mixing 1.1 g of BLIS M18 with 9.9 g of phosphate buffered saline (PBS) for 5 min and diluting by 1 in 10 with PBS
2. adding to CABK12 and BaCa agar plates
3. spotting 10 μL of foaming agent to plates and incubating at 37° C. 5% CO₂ for 24 hrs,
4. determining inhibition

TABLE 7
Inhibition of M18 and K12

| | | Lowest % | Inhibitory to BLIS |
|---|---|---|---|
| Foaming agent | Type | v/v tested | M18 and K12 |
| Polysorbate 80 | Low foaming | 0.31% | No |
| Sodium Cocyl | Low foaming | 1.25% | Yes |
| Isethionate | | | |
| Coco Glucoside | Low foaming | 0.05% | Yes |
| Decyl Glucoside | Low foaming | 0.05% | Yes |
| Lauryl glucoside | Low foaming | 5% | Yes |
| Caprylyl glucoside | Low foaming | 0.156% | Yes |
| Sodium Lauryl | High foaming | Not tested | Yes |
| Sulphate | | | |
| Standard toothpaste | n/a | | Yes |

### Example 6: Addition to Standard Toothpaste

This example was performed to demonstrate the viability of BLIS M18 and K12 in standard toothpaste formulations.

BLIS M18 and K12 were also mixed with standard Colgate® toothpaste. The M18 sample showed an instantaneous 3 log drop in BLIS M18 and no live bacteria was detected after 7 days.

Spreading of Colgate toothpaste to an M18 lawn also showed inhibition.

BLIS K12 results were similar.

### Example 7: Inhibition of M18 in Toothpaste Formulation

This example was performed to compare inhibition of other bacterial species by M18 as a raw ingredient and in the toothpaste formulation.

Raw ingredient was provided as freeze-dried cells of Streptococcus salivarius M18 cells in a lyoprotectant mix of trehalose, maltodextrin, and lactitol, suspended in PBS.

The toothpaste formulation used was B-11 as described in Example 2.

Streptococcus salivarius M18 raw ingredient product (M18 powder with trehalose/lactitol/maltodextrin);—from BLIS Technologies Ltd, New Zealand); S. mutans 10449 (ATCC 25175)—available from American Type Culture Collection (ATCC); S. pyogenes 71-698; S. pyogenes FF22; S. pyogenes W-1; A. viscosus T14; S. mutans 31c—available from BLIS Technologies Ltd on request. S. pyogenes 71-679—standard gifted from Lewis Wannamaker.

The inhibition ability of M18 in BLIS toothpaste was compared to M18 raw ingredient. Formulations had M18 counts in 1×10⁹. The formulations were measured to 0.1 g in syringes and dispensed onto the plates for producer streaks. The raw ingredient was diluted with PBS (control) was pipetted (100 μl) also had M18 counts in 1×10⁹.

Bacteriocin production was assessed using the deferred antagonism test (Tagg and Bannister 1979; Med Microbiology 12:397.). Briefly, the test strain secretes bacteriocin(s) into the agar medium and following this, various bacteriocin-susceptible (indicator) strains are applied to the bacteriocin-containing agar. If the bacteriocin inhibits the indicator strain, there is a corresponding absence of its growth on the bacteriocin agar (i.e. an inhibition zone). In this study, deferred antagonism tests were carried out with Streptococcus salivarius M18 to assess its in vitro inhibitory activity against a wide range of oral bacteria, in particular S. mutans and S. pyogenes.

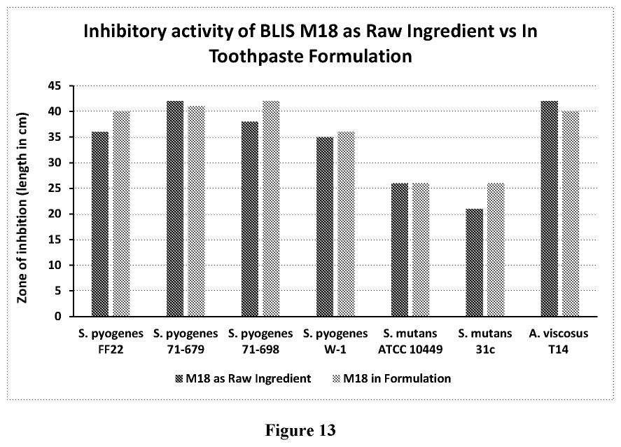

It was found that some M18 Toothpaste formulations appear to have especially inhibited the oral pathogens effectively (FIG. 13). This experiment shows that M18 in toothpaste formulations have similar or better BLIS activity compared to the PBS control.

It is not the intention to limit the scope of the invention to the abovementioned examples only. As would be appreciated by a skilled person in the art, many variations are possible without departing from the scope of the invention as set out in the appended claims.

1. An oral composition comprising a Streptococcus salivarius, a viscosity modifier, a buffering agent, and a non-aqueous carrier, wherein release of the Streptococcus salivarius from the composition at 15 minutes after being administered to an oral cavity is in the range of from about 35% to about 95%.

2. The oral composition of claim 1, wherein release of the Streptococcus salivarius from the composition at 30 minutes after being administered to an oral cavity is in the range of from about 50% to about 98%.

3. The oral composition of claim 1, wherein the Streptococcus salivarius is Streptococcus salivarius M18, Streptococcus salivarius K12, or a combination thereof.

4. The oral composition of claim 1, wherein the composition comprises the Streptococcus salivarius in an amount of about 1×10⁵ to about 1×10¹² cfu/g.

5. The oral composition of claim 1, wherein the composition comprises the viscosity modifier in an amount of about 1 to about 15% w/w.

6. The oral composition of claim 1, wherein the viscosity modifier is selected from the group consisting of hydrophobic silica, wax, ethyl cellulose, stearic acid, tapioca starch, xanthan gum, Carbopol 974p, and a combination of any two or more thereof.

7. (canceled)

8. The oral composition of claim 1, wherein the composition comprises the buffering agent in an amount of about 1 to about 30% w/w.

9. The oral composition of claim 1, wherein the buffering agent is selected from the group consisting of calcium carbonate, sodium bicarbonate, sodium chloride, sodium or potassium phosphate salts, magnesium carbonate, hydrated aluminium oxides, bentonite clay, kaolin clay, and a combination of any two or more thereof.

10-11. (canceled)

12. The oral composition of claim 1, wherein the non-aqueous carrier is selected from the group consisting of a medium chain triglyceride, triacetin, ethyl oleate, propylene glycol, vegetable oil, and a combination of any two or more thereof.

13-14. (canceled)

15. The oral composition of claim 1, further comprising an emulsifier, an antibacterial agent, a sweetener, a flavouring agent, a fluoride source, an additional probiotic, a foaming agent, a colourant, an abrasive, a whitening agent, a tooth sensitivity agent, an antioxidant, a remineralisation agent, or a combination of any two or more thereof.

16-19. (canceled)

20. The oral composition of claim 15, wherein the additional probiotic is selected from the group consisting of a Lactobacillus spp., a Bifidobacterium spp., a Streptococcus spp., a Saccharomyces spp., a Limosilactobacillus spp., a Lacticaseibacillus spp., a Ligilactobacillus spp., and a combination of any two or more thereof.

21. (canceled)

22. The oral composition of claim 1, wherein the composition has a shelf-life of at least 6 months at 25° C./60% RH.

23. The oral composition of claim 1, wherein the composition is a paste or a gel.

24. The oral composition of claim 23, wherein the composition is a gel and has a viscosity of about 35,000 to about 65,000 cp at 25° C.

25. The oral composition of claim 1, wherein the composition is a paste, and the composition has a viscosity of about 70,000 to about 2,000,000 cp at 25° C.

26-28. (canceled)

29. A toothpaste composition comprising a Streptococcus salivarius, a buffering agent, a viscosity modifier, an emulsifier, and a non-aqueous carrier, wherein the buffering agent is calcium carbonate and the viscosity modifier is hydrophobic silica.

30. The toothpaste composition of claim 29, wherein the composition comprises:
about 1×10⁵ to about 1×10¹⁰ cfu/g of Streptococcus salivarius M18,
about 1 to about 30% w/w of calcium carbonate,
about 1 to about 15% w/w of hydrophobic silica, and
about 0.1 to about 5% w/w of the emulsifier.

31. The toothpaste composition of claim 29, wherein the composition comprises:
about 1×106 to about 1×1010 cfu/g of Streptococcus salivarius M18,
about 10 to about 20% w/w of calcium carbonate,
about 3 to about 8% w/w of hydrophobic silica, and
about 1 to about 2% w/w of the emulsifier.

32-35. (canceled)

36. The toothpaste composition of claim 29, wherein release of the Streptococcus salivarius from the composition at 15 minutes after being administered to an oral cavity is in the range of from about 35% to about 95%.

37-39. (canceled)

40. A method of improving oral health in a subject, comprising applying a composition according to claim 1 to the oral cavity of the subject.

41. (canceled)
